(19) United States
(12) Patent Application Publication
(10) Pub. No.: US 2026/0184853 A1
(43) Pub. Date: Jul. 2, 2026

(54) POLYPEPTIDE ESTER ETHER AMINE RANDOM POLYMER AND PREPARATION METHOD AND APPLICATION THEREOF

(71) Applicant: ZHEJIANG UNIVERSITY, Hangzhou (CN)

(72) Inventors: JUN LING, HANGZHOU (CN); KEKE ZHANG, HANGZHOU (CN)

(21) Appl. No.: 19/328,525

(22) Filed: Sep. 15, 2025

(30) Foreign Application Priority Data
Dec. 30, 2024 (CN) 202411965817.2

(51) Int. Cl. C08G 73/02

(52) U.S. Cl. CPC C08G 73/024

(57) ABSTRACT

Disclosed are a polypeptide ester ether amine random polymer and a preparation method and application thereof. The polypeptide ester ether amine random polymer is a random copolymer of an amino acid monomer and an epoxyalkane. In the present invention, copolymerization of the amino acid monomer and the epoxyalkane monomer is achieved by a one-step monomer mixing method using a transition metal or rare earth metal salt as a catalyst. The random copolymerization product has a controllable molecular weight, a narrow molecular weight distribution, and adjustable copolymer composition. Moreover, the random copolymerization product has good solubility, biodegradability, easy functionalization and modification, good processability and good biocompatibility, and has a broad application prospect in the biomedical field.

### FIELD OF TECHNOLOGY

The present invention relates to the technical field of preparation of medical polymer materials, and specifically relates to a polypeptide ester ether amine random polymer and a preparation method and application thereof.

### BACKGROUND TECHNOLOGY

Poly(α-amino acid) (poly(amino acid), PAA), with an α-amino acid residue as its structural unit, has a structure same or similar to an α-amino acid repeating unit of a peptide chain of proteins in organisms. This structure gives it excellent biocompatibility, and PAA can be used as a biomedical polymer material. A commonly used synthetic method for the poly(α-amino acid) is a ring-opening polymerization method of α-amino acid-N-carboxylic anhydride (NCA) (Chem. Soc. Rev., 2013, 42, 7373-7390). At present, random copolymerization of amino acid monomers mainly focuses on copolymerization of different amino acid monomers, and no reports of random copolymerization with other non-nitrogen monomers have been found yet, leading to connection of all main chains of copolymerization products through rigid amide bonds (peptide bonds), thereby limiting a structural design capability for specific applications. According to the only report of polymerization of mixed monomers including lactide and γ-benzyl glutamate-NCA, the two monomers are also independently polymerized in sequence to generate a block copolymer rather than a random polymer (Macromolecules 2024, 57, 5691-5701).

Epoxyalkanes are common industrial products and basic raw materials for epoxy resins, and have been widely used in adhesives, coatings and composite materials. The epoxyalkanes are abundant in variety, low in price and prone to functionalization and modification, and are excellent monomers for copolymerization. However, no reports of copolymerization of the amino acid monomers and the epoxyalkanes have been found yet. The main reason is that nitrogen-terminated chain growth centers generated during polymerization of the amino acid monomers and oxygen-terminated centers of non-nitrogen monomers have great differences in activity, making the random copolymerization difficult to achieve.

### SUMMARY OF THE INVENTION

Aiming at the problem of difficult polymerization of amino acid monomers and non-nitrogen monomers, the present invention proposes a polypeptide ester ether amine polymer obtained by random copolymerization of an amino acid monomer and an epoxyalkane for the first time. The copolymer has a controllable molecular weight, a narrow molecular weight distribution, good solubility, biodegradability, easy functionalization and modification, good processability, good biocompatibility, and other advantages.

To achieve the above object, the present invention adopts the following technical solutions.

A polypeptide ester ether amine random polymer has the following general structural formula:

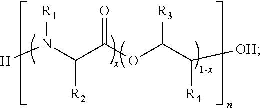

wherein R₁ and R₂ are independently selected from any one of hydrogen, a C₁-C₁₂ saturated aliphatic hydrocarbon group, a C₁-C₁₂ unsaturated aliphatic hydrocarbon group, an aromatic hydrocarbon group, an alkyl ether group, hydroxyalkyl, hydroxy-protected C₁-C₆ hydroxyalkyl, hydroxy-protected hydroxyphenylmethyl, an alkyl thioether group, alkyl indolyl, carboxy-protected C₁-C₆ carboxyalkyl, and amino-protected C₁-C₆ aminoalkyl; or R₁ and R₂ together form any one of tetrahydropyrrole and hydroxytetrahydropyrrole with a hydroxy-protected substituent;
R₃ and R₄ are independently selected from any one of hydrogen, a C₁-C₉ saturated aliphatic hydrocarbon group, and a C₁-C₉ unsaturated aliphatic hydrocarbon group; or R₃ and R₄ together form one of cyclohexane and cyclohexane substituted with methyl, ethyl, or vinyl; and x is selected from 0.30-0.99, and n is selected from 10-500.

In some embodiments, the polypeptide ester ether amine random polymer has a number-average molecular weight of 1-100 kg/mol and a molecular weight distribution of less than 2.0. In some embodiments, the polypeptide ester ether amine random polymer has the number-average molecular weight of 1-50 kg/mol.

The random copolymer provided in the present invention has an amide bond (peptide bond), an ester bond, an ether bond and an amino group simultaneously on a main chain, which can achieve improvement of performance of the copolymer in terms of water solubility, biodegradability and biocompatibility. The structure is prone to functionalization and modification, the copolymer has a controllable molecular weight, a narrow molecular weight distribution and adjustable copolymer composition, and the copolymer applicable to various scenario demands can be obtained through structural design.

The present invention further provides a method for preparing the polypeptide ester ether amine random polymer, wherein the polypeptide ester ether amine random polymer is obtained by solution polymerization of a raw material containing an α-amino acid-N-carboxylic anhydride monomer and an epoxyalkane monomer with ML as a catalyst;

In the catalyst ML, M is a transition metal or a rare earth metal element, and L is one or more of trifluoromethanesulfonate, p-toluenesulfonate, chloro, bromo, iodo, trifluoroacetate, and acetate.

In the present invention, random copolymerization of the α-amino acid-N-carboxylic anhydride monomer and the epoxyalkane is achieved by using a transition metal or rare earth metal salt as the catalyst. The transition metal salt cannot directly catalyze polymerization of the amino acid-N-carboxylic anhydride monomer, and copolymerization of the two proceeds smoothly in the presence of the epoxyalkane. According to the method, the ester bond, the ether bond and the amino group are introduced into polyamide, thereby avoiding a multi-step reaction and providing a novel and convenient synthetic method for functionalization of the polyamide. The method has universality to various α-amino acid-N-carboxylic anhydride monomers and epoxyalkanes.

Studies have found that various substituents without active hydrogen, such as alkyl, aryl, an ether, a thioether, an amide group, and an ester group, on the α-amino acid-NCA monomer, have almost no impact on ring-opening polymerization reaction performance of a five-membered ring in the monomer structure. Therefore, α-amino acid-NCA monomers with the above different substituents are all suitable for a polymerization reaction system in the present invention. In some embodiments, the α-amino acid-N-carboxylic anhydride monomer has a structure shown in formula (I-1), and the epoxyalkane monomer has a structural formula shown in formula (I-2);

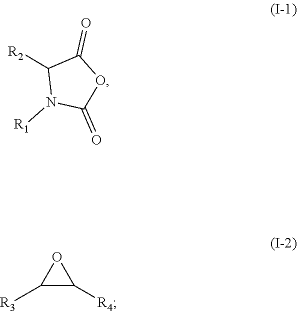

wherein R₁ and R₂ are independently selected from any one of hydrogen, a C₁-C₁₂ saturated aliphatic hydrocarbon group, a C₁-C₁₂ unsaturated aliphatic hydrocarbon group, an aromatic hydrocarbon group, an alkyl ether group, hydroxyalkyl, hydroxy-protected C₁-C₆ hydroxyalkyl, hydroxy-protected hydroxyphenylmethyl, an alkyl thioether group, alkyl indolyl, carboxy-protected C₁-C₆ carboxyalkyl, and amino-protected C₁-C₆ aminoalkyl; or R₁ and R₂ together form any one of tetrahydropyrrole and hydroxytetrahydropyrrole with a hydroxy-protected substituent;
R₃ and R₄ are independently selected from any one of hydrogen, a C₁-C₉ saturated aliphatic hydrocarbon group, and a C₁-C₉ unsaturated aliphatic hydrocarbon group; or R₃ and R₄ together form one of cyclohexane and cyclohexane substituted with methyl, ethyl, or vinyl.

In some embodiments, the α-amino acid-N-carboxylic anhydride monomer includes one or more of sarcosine-NCA, N-substituted glycine-NCA, ε-benzyloxycarbonyl lysine-NCA, ε-trifluoroacetyl lysine-NCA, γ-methyl glutamate-NCA, γ-ethyl glutamate-NCA, γ-benzyl glutamate-NCA, β-benzyl aspartate-NCA, phenylalanine-NCA, valine-NCA, leucine-NCA, isoleucine-NCA, methionine-NCA, tert-butyl serine-NCA, alanine-NCA, glycine-NCA, tryptophan-NCA, proline-NCA, threonine-NCA, O-acetylhydroxyproline-NCA, O-benzyl tyrosine-NCA, and O-benzyl dopa amino acid-NCA, etc.

In some embodiments, the N-substituted glycine-NCA has a structure shown in formula (1-3):

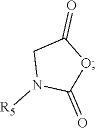

wherein R₅ is selected from a C₂-C₁₂ saturated aliphatic hydrocarbon group, a C₂-C₁₂ unsaturated aliphatic hydrocarbon group, an aromatic hydrocarbon group, an alkyl ether group, an alkyl thioether group, carboxy-protected C₁-C₆ carboxyalkyl, and amino-protected C₁-C₆ aminoalkyl.

The structural formulas of the sarcosine-NCA, ε-benzyloxycarbonyl lysine-NCA, ε-trifluoroacetyl lysine-NCA, γ-methyl glutamate-NCA, γ-ethyl glutamate-NCA, γ-benzyl glutamate-NCA, β-benzyl aspartate-NCA, phenylalanine-NCA, valine-NCA, leucine-NCA, isoleucine-NCA, methionine-NCA, tert-butyl serine-NCA, alanine-NCA, glycine-NCA, tryptophan-NCA, proline-NCA, threonine-NCA, O-acetylhydroxyproline-NCA, O-benzyl tyrosine-NCA, and O-benzyl dopa amino acid-NCA are respectively as follows:

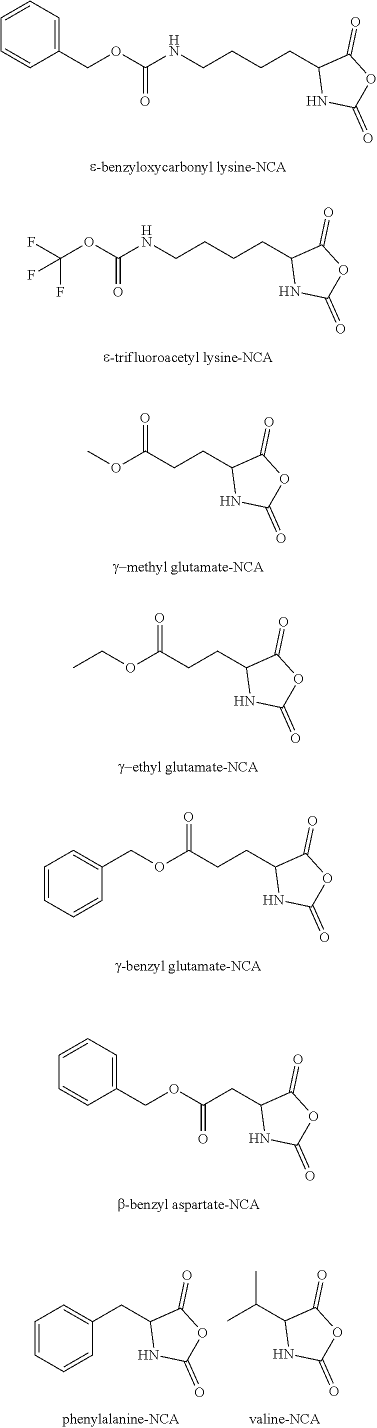

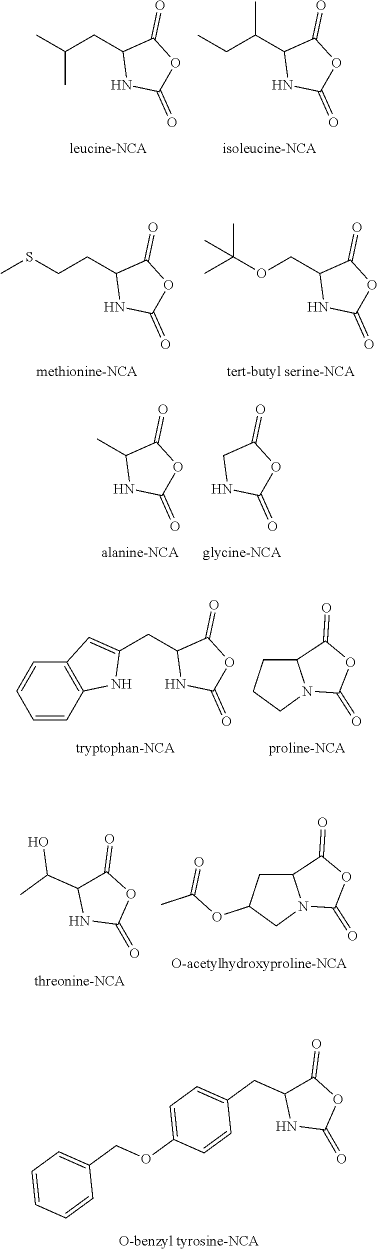

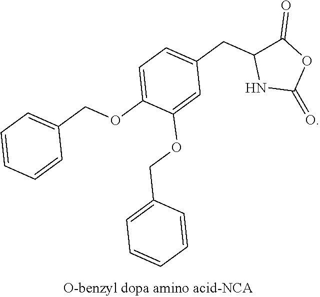 [SMILES: O=C1NC(Cc2ccc(OCc3ccccc3)c(OCc3ccccc3)c2)C(=O)O1]

In some embodiments, the epoxyalkane monomer includes one or more of ethylene oxide, 1,2-epoxypropane, butylene oxide, 1,2-epoxy-3-methoxypropane, epichlorohydrin, cyclohexene oxide, and 1,2-epoxy-4-vinylcyclohexane.

In some embodiments, the catalyst ML includes one or more of lutetium (III) trifluoromethanesulfonate, iron (III) trifluoromethanesulfonate, zinc (II) trifluoromethanesulfonate, scandium (III) trifluoromethanesulfonate, nickel (II) trifluoromethanesulfonate, copper (II) trifluoromethanesulfonate, iron (III) p-toluenesulfonate, yttrium (III) chloride, zinc p-toluenesulfonate, zinc trifluoroacetate, lutetium p-toluenesulfonate, and zinc chloride.

In some embodiments, the catalyst ML is one or more of the lutetium (III) trifluoromethanesulfonate, the iron (III) trifluoromethanesulfonate, the zinc (II) trifluoromethanesulfonate, the scandium (III) trifluoromethanesulfonate, the nickel (II) trifluoromethanesulfonate, and the copper (II) trifluoromethanesulfonate. When L is the trifluoroacetate, a reaction exhibits a higher epoxyalkane conversion rate.

In some embodiments, a molar ratio of the α-amino acid-N-carboxylic anhydride monomer to the epoxyalkane monomer is 1:0.01-1:100. Due to lower activity, the epoxyalkane monomer with a larger use amount is preferred. In some embodiments, the molar ratio of the α-amino acid-N-carboxylic anhydride monomer to the epoxyalkane monomer is 1:1.5-1:50.

In some embodiments, based on a total molar amount of the monomers, a molar ratio of the catalyst to the monomers is 1:20-1:1,000.

In some embodiments, the solution polymerization is carried out at a temperature of 10-100° C. for a time of 1-72 hours.

In some embodiments, a solvent used for the solution polymerization includes one or more of acetonitrile, methyltetrahydrofuran, dioxane, N,N-dimethylformamide, N,N-dimethylacetamide, tetramethylurea, dimethyl sulfoxide, sulfolane, nitrobenzene, benzonitrile, N-methylpyrrolidone, toluene, dichloromethane, and chloroform.

The present invention further provides use of the polypeptide ester ether amine random polymer in preparation of a water-soluble degradable medical material, for example, used as an emulsifier, a surfactant, and a solubilizer for a lipophilic drug, etc. The random copolymerization product has good solubility, biodegradability, easy functionalization and modification, good processability and good biocompatibility, can be prepared into a nanoparticle and a hydrogel, and has a broad application prospect in the biomedical field. In particular, poly(sarcosine-r-1,2-epoxy-3-methoxypropane) possesses high water solubility and biodegradability simultaneously, and is a potential high-quality substitute for medical polyethylene glycol.

Compared with the prior art, the present invention has the following beneficial effects.
(1) In the present invention, random copolymerization of the amino acid monomer and the epoxyalkane is achieved for the first time, and the polypeptide ester ether amine polymer is synthesized by polymerization using a one-step monomer mixing method. The random copolymerization product has a controllable molecular weight, a narrow molecular weight distribution, and adjustable copolymer composition. More importantly, the random copolymerization product has good solubility, biodegradability, easy functionalization and modification, good processability and good biocompatibility, can be prepared into a nanoparticle and a hydrogel, and has a broad application prospect in the biomedical field.
(2) The method for preparing the polypeptide ester ether amine polymer in the present invention has wide sources of raw materials, simple operation and high universality, and is suitable for most amino acids and epoxyalkanes, wherein the amino acids have biomass sources, the epoxyalkanes are petroleum industry products, and both have the characteristics of being inexpensive, readily available, and abundant in variety. The method is suitable for large-scale industrial production.

### BRIEF DESCRIPTION OF THE DRAWINGS

FIG. 1 is a nuclear magnetic resonance spectrum diagram of a random copolymer poly(sarcosine-r-cyclohexene oxide) prepared in Example 1.

FIG. 2 is a nuclear magnetic resonance spectrum diagram of a random copolymer poly(sarcosine-r-1,2-epoxy-3-methoxypropane) prepared in Example 2.

FIG. 3 is a gel permeation chromatography curve diagram of an enzymatic degradation experiment of a random copolymer prepared in Example 2.

FIG. 4 is a dynamic light scattering diagram of an aqueous solution of a random copolymer prepared in Example 11.

### DETAILED DESCRIPTION OF THE EMBODIMENTS

To achieve the object, technical solutions and advantages of the present invention more clearly understood, the present invention is further illustrated in detail below in conjunction with examples. It should be understood that the specific examples described herein are merely used to explain the present invention, rather than to limit the present invention. Modifications or equivalent substitutions made by those skilled in the art based on understanding of the technical solutions of the present invention without departing from the spirit and scope of the technical solutions of the present invention shall be encompassed within the scope of protection of the present invention.

In the following specific embodiments, a molecular weight and molecular weight distribution of a polymer are determined by gel permeation chromatography (SEC, Waters 1515) (hexafluoroisopropanol containing 3 mg/L of potassium trifluoroacetate, 40° C., at a flow rate of 0.8 mL/min); and a nuclear magnetic resonance hydrogen spectrum (¹H NMR) is determined on a Bruker Avance DMX 400 instrument, using deuterated chloroform or deuterated dimethyl sulfoxide as a solvent and tetramethylsilane as an internal standard. Unless otherwise specified, all raw materials are purchased from the market.

A hydrodynamic diameter of a polymeric nanomicelle in a solution is detected through a Zetasizer Nano Series (Malvern Instruments) detector, with a measurement wavelength of 657 nm and a fixed angle of 90°, and each sample is tested in parallel for 3 times.

### Example 1

200 mg of sarcosine-NCA (1.74 mmol) and 10.8 mg of lutetium (III) trifluoromethanesulfonate (0.0174 mmol) were dissolved in 1.4 mL of acetonitrile, and 341 mg of cyclohexene oxide (3.48 mmol) was finally added, where a molar ratio of the sarcosine-NCA, the cyclohexene oxide and the catalyst was 100:200:1. After vibration and even shaking, a mixture was placed in an oil bath to carry out a reaction at 60° C. for two days. A polymerization solution was subjected to precipitation in ethyl ether, filtration, and vacuum drying to a constant weight to obtain a polypeptide ester ether amine copolymer.

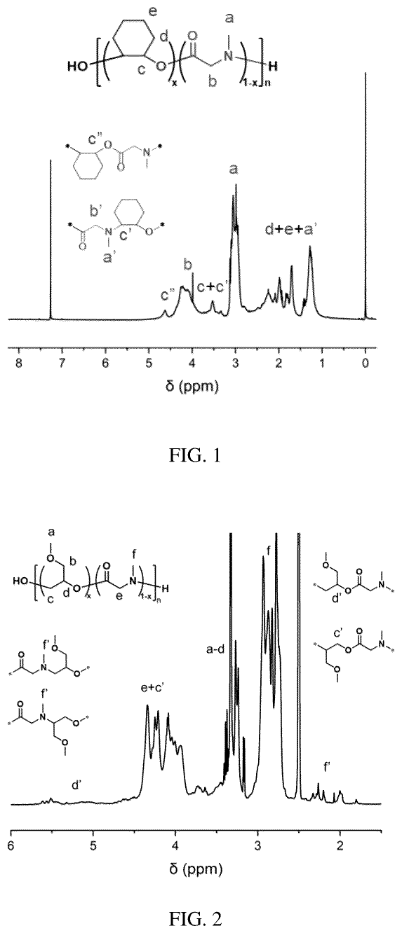

According to testing, the polymerization product has a number-average molecular weight of 11.5 kg/mol as determined by size exclusion chromatography (SEC), a cyclohexene oxide mole content of 0.27, a sarcosine residue mole content of 0.73, a sarcosine-NCA monomer conversion rate of 99%, and a molecular weight distribution of 1.64. A ¹H NMR spectrum (CDCl₃) of the copolymer is shown in FIG. 1. Various signal attributions are clear, and structural characterization is clear, indicating that the obtained product is a polypeptide ester ether amine polymer obtained by copolymerization of the sarcosine and the cyclohexene oxide.

### Example 2

According to a preparation process in Example 1, a difference is only that the cyclohexene oxide used was replaced with an equivalent molar amount of 1,2-epoxy-3-methoxypropane to achieve copolymerization of the sarcosine-NCA and the 1,2-epoxy-3-methoxypropane. A molar ratio of the sarcosine-NCA, the 1,2-epoxy-3-methoxypropane and the catalyst was 100:200:1.

According to testing, a polymerization product prepared in the present example has a number-average molecular weight of 9.6 kg/mol as determined by SEC, a 1,2-epoxy-3-methoxypropane mole content of 0.14, a sarcosine residue mole content of 0.86, a molecular weight distribution of 1.33, and an amino acid monomer conversion rate of 99%. A ¹H NMR spectrum (DMSO-d6) of a copolymer is shown in FIG. 2. All signal attributions are clear, and structural characterization is clear, indicating that the obtained product was a polypeptide ester ether amine product of the sarcosine-NCA and the 1,2-epoxy-3-methoxypropane.

According to testing, the copolymer has excellent water solubility (>800 g/L) and biological lipase degradability, and is a potential good substitute for medical polyethylene glycol.

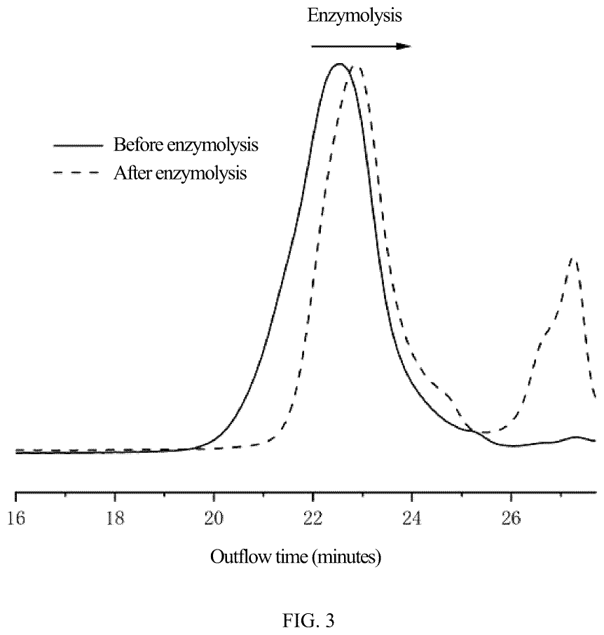

30 mg of the copolymer prepared in the present example and 15 mg of porcine pancreatic lipase were dissolved in 2.7 mL of a PBS solution (0.1 mol/L, pH=7.5), then 0.3 mL of ethanol was added, and a mixture was incubated at 37° C. for 15 days, during which the number-average molecular weight was decreased from 9.6 kg/mol to 3.2 kg/mol. A gel permeation chromatography curve of an enzymatic degradation experiment is shown in FIG. 3. In the prior art, poly(amino acids) and polyethers themselves do not have the possibility of being degraded by lipase. The copolymer prepared in the present invention has high water solubility and lipase degradability, and can be used as a substitute for polyethylene glycol to prepare medical auxiliary materials with more excellent performance, water solubility and biodegradability.

### Example 3

According to a preparation process in Example 1, differences are only that the sarcosine-NCA used was replaced with γ-benzyl glutamate-NCA to achieve copolymerization of the γ-benzyl glutamate-NCA and the cyclohexene oxide, and a molar ratio of the γ-benzyl glutamate-NCA, the cyclohexene oxide and the catalyst was 30:60:1.

According to testing, a polymerization product prepared in the present example had a number-average molecular weight of 1.5 kg/mol as determined by SEC, a cyclohexene oxide mole content of 0.49, a γ-benzyl glutamate residue mole content of 0.51, a molecular weight distribution of 1.32, and an amino acid monomer conversion rate of 99%.

### Example 4

According to a preparation process in Example 1, a difference is only that the cyclohexene oxide used was replaced with epoxypropane to achieve copolymerization of the sarcosine-NCA and the epoxypropane. A molar ratio of the sarcosine-NCA, the epoxypropane and the catalyst was 100:200:1.

According to testing, a polymerization product prepared in the present example had a number-average molecular weight of 4.4 kg/mol as determined by SEC, an epoxypropane mole content of 0.11, a sarcosine residue mole content of 0.89, a molecular weight distribution of 1.09, and an amino acid monomer conversion rate of 99%.

### Example 5

According to a preparation process in Example 1, a difference is only that the sarcosine-NCA used was replaced with ε-benzyloxycarbonyl lysine-NCA to achieve copolymerization of the ε-benzyloxycarbonyl lysine-NCA and the cyclohexene oxide. A molar ratio of the ε-benzyloxycarbonyl lysine-NCA, the cyclohexene oxide and the catalyst was 100:200:1.

According to testing, a polymerization product prepared in the present example had a number-average molecular weight of 1.6 kg/mol as determined by SEC, a cyclohexene oxide mole content of 0.15, an ε-benzyloxycarbonyl lysine residue mole content of 0.85, a molecular weight distribution of 1.48, and an amino acid monomer conversion rate of 99%.

### Example 6

According to a preparation process in Example 1, differences are only that N-benzyl glycine-NCA was used to undergo copolymerization with the cyclohexene oxide, and a molar ratio of the N-benzyl glycine-NCA, the cyclohexene oxide and the catalyst was 30:90:1.

According to testing, a polymerization product prepared in the present example had a number-average molecular weight of 1.5 kg/mol as determined by SEC, a cyclohexene oxide mole content of 56%, an N-benzyl glycine residue mole content of 44%, a molecular weight distribution of 1.20, and an amino acid monomer conversion rate of 99%.

### Example 7

According to a preparation process in Example 1, differences are only that an equivalent molar amount of zinc trifluoroacetate was used to replace the lutetium (III) trifluoromethanesulfonate as the catalyst, and a random copolymer was prepared by solution polymerization at a temperature of 80° C. for a reaction time of two days.

According to testing, the polymerization product prepared in the present example had a number-average molecular weight of 9.0 kg/mol as determined by SEC, a cyclohexene oxide mole content of 0.07, a sarcosine residue mole content of 0.93, a molecular weight distribution of 1.16, and an amino acid monomer conversion rate of 99%.

### Example 8

According to a preparation process in Example 1, differences are only that an equivalent molar amount of iron (III) p-toluenesulfonate was used to replace the lutetium (III) trifluoromethanesulfonate as the catalyst, and a random copolymer was prepared by solution polymerization at a temperature of 80° C. for a reaction time of two days.

According to testing, the polymerization product prepared in the present example had a number-average molecular weight of 16.6 kg/mol as determined by SEC, a cyclohexene oxide mole content of 0.10, a sarcosine residue mole content of 0.90, a molecular weight distribution of 1.55, and an amino acid monomer conversion rate of 99%.

### Example 9

According to a preparation process in Example 1, differences are only that an equivalent molar amount of scandium (III) trifluoromethanesulfonate was used to replace the lutetium (III) trifluoromethanesulfonate as the catalyst, and a random copolymer was prepared by solution polymerization at a temperature of 80° C. for a reaction time of two days.

According to testing, the polymerization product prepared in the present example had a number-average molecular weight of 11.1 kg/mol as determined by SEC, a cyclohexene oxide mole content of 0.12, a sarcosine residue mole content of 0.88, a molecular weight distribution of 1.88, and an amino acid monomer conversion rate of 99%.

### Example 10

According to a preparation process in Example 1, differences are only that an equivalent molar amount of iron (III) trifluoromethanesulfonate was used to replace the lutetium (III) trifluoromethanesulfonate as the catalyst, and a random copolymer was prepared by solution polymerization at a temperature of 80° C. for a reaction time of two days.

According to testing, the polymerization product prepared in the present example had a number-average molecular weight of 19.8 kg/mol as determined by SEC, a cyclohexene oxide mole content of 0.13, a sarcosine residue mole content of 0.87, a molecular weight distribution of 1.53, and an amino acid monomer conversion rate of 99%.

### Example 11

According to a preparation process in Example 1, a difference is only that the molar ratio of the sarcosine-NCA, the cyclohexene oxide and the catalyst was 100:400:1.

According to testing, a polymerization product had a number-average molecular weight of 8.9 kg/mol as determined by size exclusion chromatography (SEC), a cyclohexene oxide mole content of 0.34, a sarcosine residue mole content of 0.66, a sarcosine-NCA monomer conversion rate of 99%, and a molecular weight distribution of 1.77.

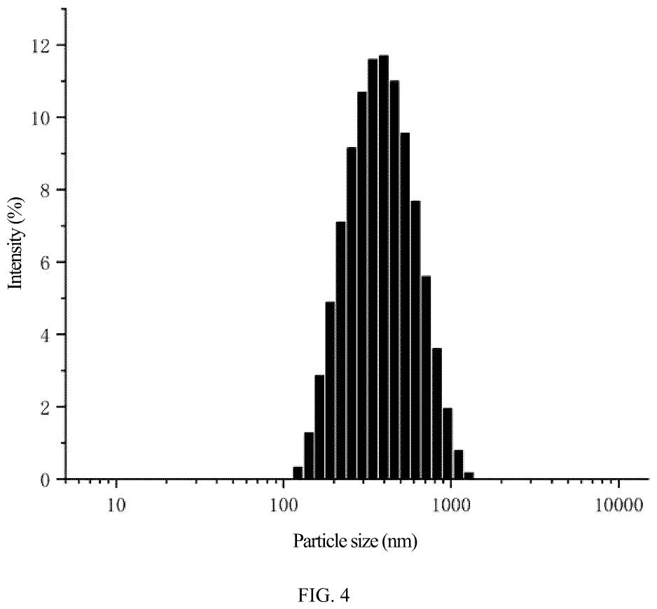

As shown in FIG. 4, the polymerization product (hydrophilic-lipophilic balance value (HLB)=11.7) can form a nanoparticle with a particle size of approximately 400 nm in water and can be used as a solubilizer for a lipophilic drug.

The above examples represent preferred embodiments of the present invention, but the embodiments of the present invention are not limited to the above examples. Any other alterations, modifications, substitutions, combinations and simplifications made without departing from the spirit, essence and principles of the present invention shall be considered as equivalent replacement modes and are all included within the scope of protection of the present invention.

1. A polypeptide ester ether amine random polymer, having the following general structural formula: 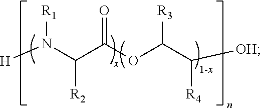
wherein R₁ and R₂ are independently selected from any one of hydrogen, a C₁-C₁₂ saturated aliphatic hydrocarbon group, a C₁-C₁₂ unsaturated aliphatic hydrocarbon group, an aromatic hydrocarbon group, an alkyl ether group, hydroxyalkyl, hydroxy-protected C₁-C₆ hydroxyalkyl, hydroxy-protected hydroxyphenylmethyl, an alkyl thioether group, alkyl indolyl, carboxy-protected C₁-C₆ carboxyalkyl, and amino-protected C₁-C₆ aminoalkyl; or R₁ and R₂ together form any one of tetrahydropyrrole and hydroxytetrahydropyrrole with a hydroxy-protected substituent;
R₃ and R₄ are independently selected from any one of hydrogen, a C₁-C₉ saturated aliphatic hydrocarbon group, and a C₁-C₉ unsaturated aliphatic hydrocarbon group; or R₃ and R₄ together form one of cyclohexane and cyclohexane substituted with methyl, ethyl, or vinyl; and x is selected from 0.30-0.99, and n is selected from 10-500.

2. The polypeptide ester ether amine random polymer according to claim 1, wherein the polypeptide ester ether amine random polymer has a number-average molecular weight of 1-100 kg/mol and a molecular weight distribution of less than 2.0.

3. A method for preparing the polypeptide ester ether amine random polymer according to claim 1, wherein the polypeptide ester ether amine random polymer is obtained by solution polymerization of a raw material comprising an α-amino acid-N-carboxylic anhydride monomer and an epoxyalkane monomer with ML as a catalyst; and
in the catalyst ML, M is a transition metal or rare earth metal element, and L is one or more of trifluoromethanesulfonate, p-toluenesulfonate, chloro, bromo, iodo, trifluoroacetate, and acetate.

4. The method for preparing the polypeptide ester ether amine random polymer according to claim 3, wherein the α-amino acid-N-carboxylic anhydride monomer has a structure shown in formula (I-1), and the epoxyalkane monomer has a structural formula shown in formula (I-2); 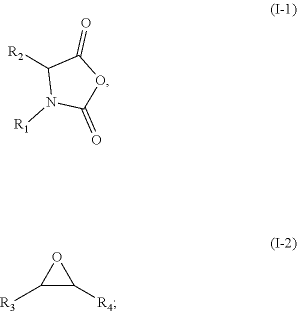
wherein R₁ and R₂ are independently selected from any one of hydrogen, a C₁-C₁₂ saturated aliphatic hydrocarbon group, a C₁-C₁₂ unsaturated aliphatic hydrocarbon group, an aromatic hydrocarbon group, an alkyl ether group, hydroxyalkyl, hydroxy-protected C₁-C₆ hydroxyalkyl, hydroxy-protected hydroxyphenylmethyl, an alkyl thioether group, alkyl indolyl, carboxy-protected C₁-C₆ carboxyalkyl, and amino-protected C₁-C₆ aminoalkyl; or R₁ and R₂ together form any one of tetrahydropyrrole and hydroxytetrahydropyrrole with a hydroxy-protected substituent;
R₃ and R₄ are independently selected from any one of hydrogen, a C₁-C₉ saturated aliphatic hydrocarbon group, and a C₁-C₉ unsaturated aliphatic hydrocarbon group; or R₃ and R₄ together form one of cyclohexane and cyclohexane substituted with methyl, ethyl, or vinyl.

5. The method for preparing the polypeptide ester ether amine random polymer according to claim 3, wherein the α-amino acid-N-carboxylic anhydride monomer comprises one or more of sarcosine-NCA, N-substituted glycine-NCA, ε-benzyloxycarbonyl lysine-NCA, ε-trifluoroacetyl lysine-NCA, T-methyl glutamate-NCA, T-ethyl glutamate-NCA, γ-benzyl glutamate-NCA, O-benzyl aspartate-NCA, phenylalanine-NCA, valine-NCA, leucine-NCA, isoleucine-NCA, methionine-NCA, tert-butyl serine-NCA, alanine-NCA, glycine-NCA, tryptophan-NCA, proline-NCA, threonine-NCA, O-acetylhydroxyproline-NCA, O-benzyl tyrosine-NCA, and O-benzyl dopa amino acid-NCA;
the N-substituted glycine-NCA has a structure shown in formula (I-3): 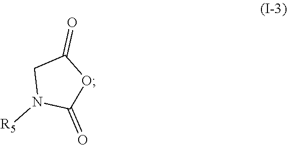
wherein R₅ is selected from a C₂-C₁₂ saturated aliphatic hydrocarbon group, a C₂-C₁₂ unsaturated aliphatic hydrocarbon group, an aromatic hydrocarbon group, an alkyl ether group, an alkyl thioether group, carboxy-protected C₁-C₆ carboxyalkyl, and amino-protected C₁-C₆ aminoalkyl;
and/or, the epoxyalkane monomer comprises one or more of ethylene oxide, 1,2-epoxypropane, butylene oxide, 1,2-epoxy-3-methoxypropane, epichlorohydrin, cyclohexene oxide, and 1,2-epoxy-4-vinylcyclohexane.

6. The method for preparing the polypeptide ester ether amine random polymer according to claim 3, wherein the catalyst ML comprises one or more of lutetium (III) trifluoromethanesulfonate, iron (III) trifluoromethanesulfonate, zinc (II) trifluoromethanesulfonate, scandium (III) trifluoromethanesulfonate, nickel (II) trifluoromethanesulfonate, copper (II) trifluoromethanesulfonate, iron (III) p-toluenesulfonate, yttrium (III) chloride, zinc p-toluenesulfonate, zinc trifluoroacetate, lutetium p-toluenesulfonate, and zinc chloride.

7. The method for preparing the polypeptide ester ether amine random polymer according to claim 3, wherein a molar ratio of the α-amino acid-N-carboxylic anhydride monomer to the epoxyalkane monomer is 1:0.01-1:100.

8. The method for preparing the polypeptide ester ether amine random polymer according to claim 3, wherein based on a total molar amount of the monomers, a molar ratio of the catalyst to the monomers is 1:20-1:1,000.

9. The method for preparing the polypeptide ester ether amine random polymer according to claim 3, wherein the solution polymerization is carried out at a temperature of 10-100° C. for a time of 1-72 hours.

10. A method for preparing a water-soluble degradable medical material comprising the step of utilizing the polypeptide ester ether amine random polymer according to claim 1.
